(19) United States
(12) Patent Application Publication
(10) Pub. No.: US 2025/0303194 A1
(43) Pub. Date: Oct. 2, 2025

(54) RADIONUCLIDE SOURCE CLIP REMOVAL SYSTEM

(71) Applicant: ARTBIO, Inc., Cambridge, MA (US)

(72) Inventors: Gjermund Fjeld Olsen, Fjerdingby (NO); Tanja Aarvak, Oslo (NO); Judit Tjelmeland Oestby, Kraakstad (NO); Olga Salina, Ski (NO); John Emmanuel Aristidou, Cambridge (GB)

(73) Assignee: ARTBIO, Inc., Cambridge, MA (US)

(21) Appl. No.: 19/090,754

(22) Filed: Mar. 26, 2025

Related U.S. Application Data
(60) Provisional application No. 63/571,561, filed on Mar. 29, 2024.

(51) Int. Cl. A61N 5/10; A61B 17/08; A61B 17/10; A61B 17/34

(52) U.S. Cl. CPC A61N 5/1007; A61B 17/083; A61B 17/10; A61B 17/3468; A61N 2005/1019

(57) ABSTRACT

Some aspects are generally related to radionuclide source clip handling systems configured for use with a radionuclide source holder. In some embodiments, radionuclide source clip handling system is configured to remove a radionuclide source clip from a distal end portion of the radionuclide source holder to expose a radionuclide source, e.g., to utilize the radionuclide source to generate progeny radionuclides. Still other aspects are generally directed to related methods of use of the radionuclide source clip and radionuclide source clip handling system.

### CROSS REFERENCE TO RELATED APPLICATIONS

This Application claims priority under 35 U.S.C. § 119(e) to U.S. Provisional Application No. 63/571,561, filed Mar. 29, 2024, which is incorporated herein by reference in its entirety for all purposes.

### FIELD

Disclosed embodiments are related to a radionuclide source clip handling system, which may be used to remove radionuclide source clips in some embodiments, that is configured for use with a radionuclide source holder, and related methods of use.

### BACKGROUND

Radionuclides such as lead 212 (²¹²Pb) may be used in various applications. For example, ²¹²Pb may be used as a therapeutic in radiation treatments for various health conditions, including various cancers. Lead 212 may be formed as a progeny radionuclide in the decay chain of a parent radionuclide such as thorium, radon, or radium.

### SUMMARY

In some embodiments, a radionuclide source clip handling system comprises a first clip housing portion; a second clip housing portion, wherein the first clip housing portion is configured to be moved away from and towards the second clip housing portion; a first opening formed in the first clip housing portion; and a radionuclide source clip interface configured to retain a radionuclide source clip from a distal end portion of a radionuclide source holder positioned in the first opening, wherein the radionuclide source clip interface is connected to the first clip housing portion such that moving the first clip housing portion from an initial configuration to a retracted configuration is configured to remove the radionuclide source clip from the radionuclide source holder.

In some embodiments, a method of removing a radionuclide source interface comprises inserting a distal end portion of a radionuclide source holder into a first opening of a first clip housing portion; engaging a radionuclide source clip of the radionuclide source holder with a radionuclide source clip interface; and moving the first clip housing portion from an initial configuration to a retracted configuration to remove the radionuclide source clip from the radionuclide source holder.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a diagram showing decay pathways between radionuclides, according to some embodiments;

FIG. 2 is an image of a radionuclide source clip handling system, a transport container, and a radionuclide generator, according to some embodiments;

FIG. 3A is a cross-sectional diagram of a radionuclide source holder in an extended configuration, according to some embodiments;

FIG. 3B is a cross-sectional diagram of a radionuclide source holder in a retracted configuration, according to some embodiments;

FIG. 4A is an exploded view of a radionuclide source clip, according to some embodiments;

FIG. 4B is a perspective view of a radionuclide source clip in an unengaged configuration relative to a radionuclide source holder, according to some embodiments;

FIG. 4C is a perspective view of a radionuclide source clip in an engaged configuration relative to a radionuclide source holder, according to some embodiments;

FIG. 4D is a side view of a radionuclide source clip in an engaged configuration relative to a radionuclide source holder, according to some embodiments;

FIG. 5A is a perspective view of a radionuclide source clip handling system, according to some embodiments;

FIG. 5B is a perspective view of a radionuclide source clip handling system, according to some embodiments;

FIG. 6 is a perspective view of a distal end portion of a radionuclide source holder inserted into a radionuclide source clip handling system, according to some embodiments;

FIG. 7A is a cross-sectional view of a distal end portion of a radionuclide source holder inserted into a radionuclide source clip handling system in an initial configuration, according to some embodiments;

FIG. 7B is a cross-sectional view of a distal end portion of a radionuclide source

holder inserted into a radionuclide source clip handling system in a retracted configuration, according to some embodiments;

FIG. 7C is a cross-sectional view of a radionuclide source clip handling system in a sealed configuration, according to some embodiments;

FIG. 7D is a perspective view of a radionuclide source clip handling system in a sealed configuration, according to some embodiments;

FIG. 7E is a perspective view of a radionuclide source clip handling system with the sealed housing including a clip sealed therein removed from the radionuclide source clip handling system, according to some embodiments;

FIG. 8A is a cutaway view of a radionuclide source clip interface of a radionuclide source clip handling system, according to some embodiments;

FIG. 8B is a cutaway view of a radionuclide source clip interface of a radionuclide source clip handling system engaged with a radionuclide source clip, according to some embodiments; and

FIG. 9 is a method flow diagram detailing of a method for using a radionuclide source clip handling system, according to some embodiments.

### DETAILED DESCRIPTION

Radionuclides may be used for a variety of applications in such fields as medicine, biology, physics, and other industries. Some radionuclides possess relatively short half-lives and thus may be appropriate for use in various medical applications, such as targeted alpha-particle therapy (TAT) where the relatively short half-lives are preferable for treatment of certain conditions (e.g., any of various cancers such as prostate or carcinoid cancers) while limiting the time of exposure of a patient to radioactivity and accordingly minimizing side effects from the radionuclide treatment. Short half-life radionuclides (e.g., lead 212, ²¹²Pb) may be difficult to ship and/or store because the short half-life may lead to quick decay into undesirable daughter, granddaughter, or other progeny radionuclides during shipping or storage before utilized as desired. In contrast, a parent, grandparent, or other precursor radionuclide to the short half-life radionuclide may have a relatively long half-life, such that the precursor radionuclide may be shipped and/or stored for longer periods. To facilitate shipping and/or storage of radionuclide sources the inventors have developed radionuclide source clips that may be used to help maintain a position and/or seal a radionuclide source within a radionuclide source holder during transportation and handling.

While the above noted radionuclide source clips may facilitate storage and/or transport of a radionuclide source within a radionuclide source holder, such clips also inhibit and/or prevent the ability to use the radionuclide source holder and/or the radionuclide source disposed therein. That is, when the radionuclide source clip is engaged with the radionuclide source holder, the radionuclide source may not be exposed. Further, the removal of such a clip from a radionuclide source holder when it is desired to use the holder may be difficult due to radionuclide holders and generators often being manipulated using shielded glove boxes, tongs, robotic manipulators, and/or with other methods and systems that make manipulation and removal of structures from a radionuclide source holder difficult.

In view of the above, the inventors have recognized the benefits associated with a radionuclide source clip handling system that facilitates the removal of a radionuclide source clip from a radionuclide source holder. Advantageously, the radionuclide source clip handling system may be easily manipulated using work processes and systems typically used in the manipulation of radioactive materials.

It will also be appreciated that the attachment of such a radionuclide source clip to a radionuclide source holder may be desirable in some instances and that such an operation may be similarly difficult due to radionuclide holders and generators often being manipulated using shielded glove boxes, tongs, robotic manipulators, and/or with other methods and systems suitable for manipulating radionuclide source containing systems. While generally described in the context of removing the radionuclide source clip from the radionuclide source holder, the radionuclide source clip handling systems described herein may advantageously also facilitate the attachment of the radionuclide source clip onto the radionuclide source holder by performing the discussed operations in the reverse manner.

In some embodiments of a radionuclide source clip handling system, a distal end portion of radionuclide source holder with a radionuclide source clip disposed thereon may be inserted into and supported within the radionuclide source clip handling system. A radionuclide source clip handling system may include a radionuclide source clip interface that is configured to be selectively engaged with the radionuclide source clip of the radionuclide source holder when the holder is inserted into the system and the first clip housing portion is in an initial configuration. The radionuclide source interface may be configured to selectively retain and remove the radionuclide source clip from a radionuclide source holder to expose the radionuclide source disposed within the holder. For example, the radionuclide source interface may be configured to be moved from the initial configuration to one or more other configurations to remove the radionuclide source clip from the radionuclide source holder as elaborated on further below. For example, following engagement of the radionuclide source clip by the radionuclide source clip interface, which may be associated with a first clip housing portion, the radionuclide source clip interface may move from the initial configuration to a retracted configuration to remove and space the clip from the holder to expose the radionuclide source disposed in the holder. After removing the radionuclide source clip, the radionuclide source holder may be removed from the first clip housing portion of the system, and then the first clip housing portion along with the associated radionuclide source clip interface may be moved from the retracted configuration to a sealed configuration where the first clip housing portion interfaces with a second clip housing portion to form a sealed volume. In the sealed configuration, the radionuclide source clip may be retained in the sealed volume formed by the first and second clip housing portions.

Using such a radionuclide source clip handling system may lead to simple and reproducible removal of a radionuclide source clip. In some such embodiments, after removing the radionuclide source clip, the radionuclide source clip handling system may facilitate the disposal of the radionuclide source clip. In such a manner, the direct handling of components that may be exposed to the radionuclide source (and thus may be coated in radionuclides) may be avoided.

While the radionuclide source clip handling system is described in the context of a radionuclide source holder configured for use with radionuclide sources having long half-lives, it should further be understood that the radionuclide source holder, radionuclide source clip, and components of the radionuclide source clip handling system may be configured to remove a clip from a radionuclide source holder containing any type of a variety of radionuclide sources as the disclosure is not so limited.

The various components of the systems described herein, e.g., the housing (e.g., a first and/or second clip housing portion) and/or the radionuclide source clip of the radionuclide source clip handling system, the radionuclide source clip, and/or the radionuclide source holder, may be made of any of a variety of materials that are resistant to radiation from the radionuclide source that may be contained in the radionuclide source holder. This avoids degradation of the components by any radiation that may occur when the radionuclide source is exposed following removal of the radionuclide source clip from the radionuclide source holder. Non-limiting examples of materials from which the various components may independently be constructed include metals, ceramics, plastics, polymers, rubbers, or the like. Other materials are also possible. In some embodiments, the materials of some or all of the components may be at least partially constructed of materials that shield radiation such that they shield at least a portion of radiation that is emitted from a radionuclide source. For example, in some embodiments, the material of the various components may include lead, tungsten, and/or other suitable shielding materials.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

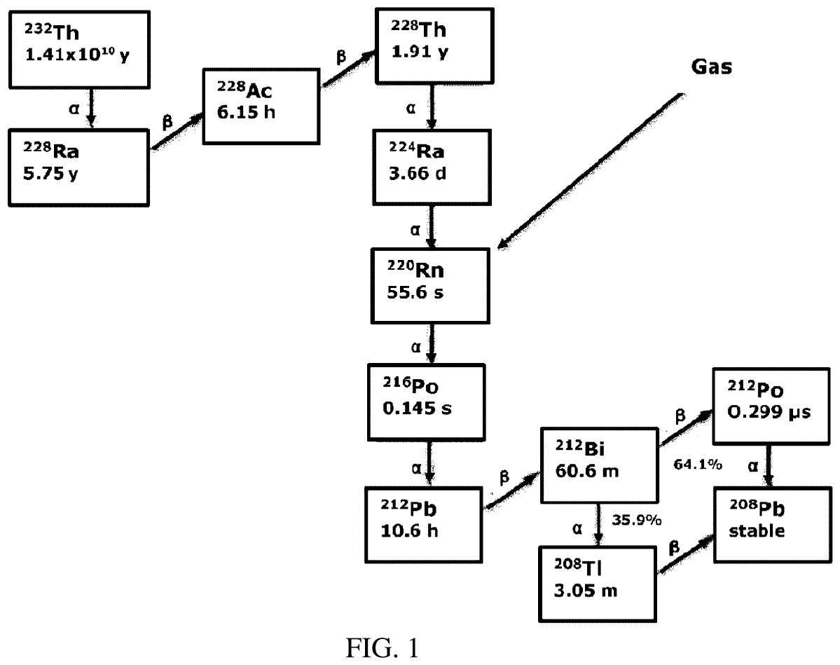

FIG. 1 depicts a diagram of an exemplary thorium series decay chain beginning with thorium 232 (²³²Th). The half-life of each radionuclide in the decay chain is noted in the figure. Note that the radon 220 radionuclide (²²⁰Rn) rapidly decays (e.g., a half-life of 55.6 seconds) into polonium 216 (²¹⁶Po), which further quickly decays (e.g., a half-life of 0.145 seconds) into a lead 212 radionuclide (²¹²Pb). The approximately 10.6 hour half-life of ²¹²Pb may be desirable for various applications. It will further be noted from FIG. 1 that ²²⁰Rn may be a gas at ambient pressure and temperature. Accordingly, it will be appreciated that when a source including a precursor radionuclide to ²²⁰Rn (e.g., ²²⁴Ra, ²²⁸Th, ²²⁸Ac, ²²⁸Ra, or ²³²Th) is exposed to a portion of a container (e.g., an interior surface), the precursor radionuclide may decay into the gaseous ²²⁰Rn. The gaseous ²²⁰Rn may then decay into (e.g., via ²¹⁶Po) ²¹²Pb and may deposit onto the exposed portion of the container as ²¹²Pb.

Note that the materials described in FIG. 1 are exemplary, and that the radionuclide source holder, radionuclide source clip, and radionuclide source clip handling system are suitable for use with any of a variety of radionuclide source materials that may be transported in a radionuclide source holder and/or used to generate progeny radionuclides in a radionuclide generator.

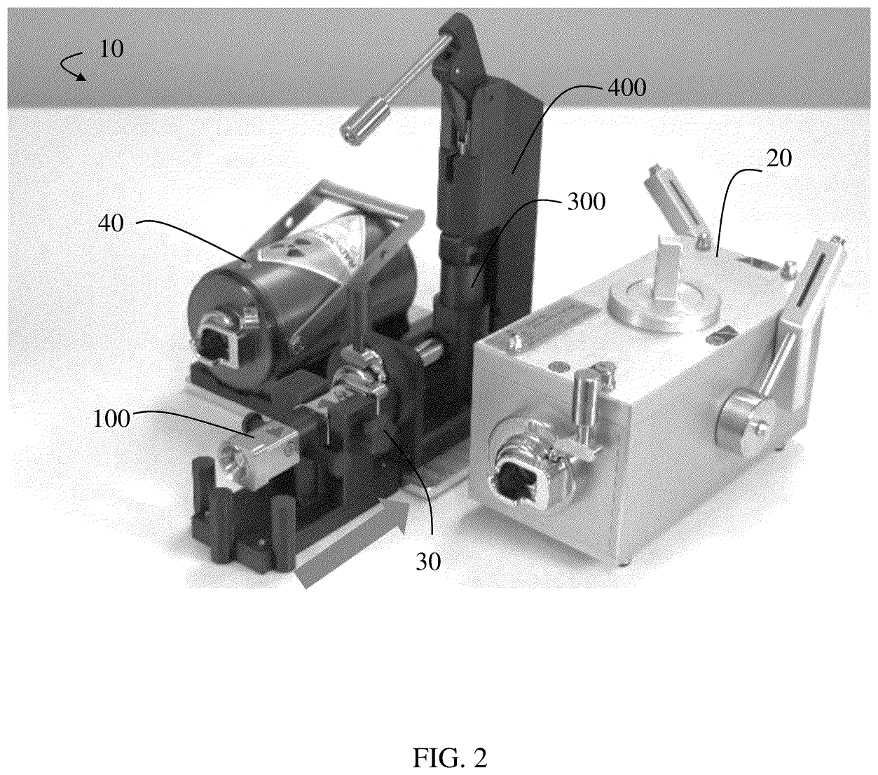

FIG. 2 depicts embodiments of various components of a radionuclide handling system 10. The radionuclide handling system 10 includes a radionuclide generator 20, a support 30, a transport container 40, and a radionuclide source holder 100 that may be supported in a desired location and orientation by a support 30. The support 30 may include appropriate features that are configured to hold the radionuclide source holder 100 in an appropriate pose such that a portion of the radionuclide source holder 100 may interact with a radionuclide source clip handling system 300 to remove a clip from the radionuclide source holder 100, as described in more detail elsewhere herein. In some instances, the radionuclide source clip handling system 300 may be engaged with a manipulator 400 which may be used to manually or automatically actuate the radionuclide source clip handling system 300. The system may also include a radionuclide generator 20 that may also be configured to accept the radionuclide source holder 100 and to expose a radionuclide source contained in the radionuclide source holder 100 (e.g., to utilize progeny radionuclides produced therefrom). The radionuclide handling system 10 may also include a radionuclide source transport container 40 that is configured to receive at least a portion of the radionuclide source holder 100 disposed therein for transporting the radionuclide source holder 100.

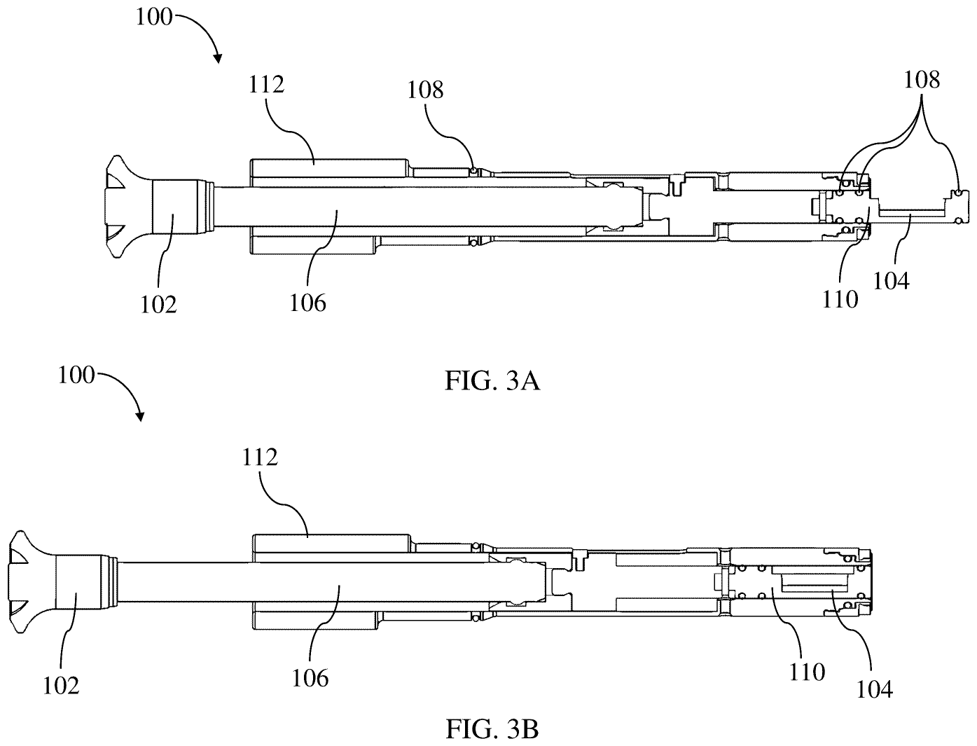

FIGS. 3A and 3B depict cross-sections of the radionuclide source holder 100. FIG. 3A depicts the source 104 in an extended configuration, while FIG. 3B depicts the source 104 in a retracted configuration. A radionuclide source holder 100 may include a radionuclide source receptacle 110 configured to receive a precursor radionuclide source 104 disposed therein, for example by including an opening, slot, cavity, divot, channel, rod, or other receptacle sized and shaped to receive and retain the radionuclide source 104. It will be appreciated that a source may be formed in any appropriate regular or irregular geometry, including a cylinder, a disc, a tablet, a block, a chip, a sphere, a sheet, a plate, a ball, a rod, or any other appropriate geometry capable of being positioned in the radionuclide source receptacle 110.

Similarly, although the source receptacle 110 is depicted as a cavity or hollowed-out portion of the source holder 100, it will be appreciated that a source holder 100 may include any source receptacle 110 appropriately formed to receive a correspondingly shaped source. For example, in some embodiments, the source holder may be configured to engage with and retain a source around a perimeter and/or periphery of the source while opposing sides of the source are exposed. For example, a through hole including two opposing openings may be formed in an exposable portion of a source holder 100 with a source disposed in the through hole. Thus, it should be understood that the currently disclosed systems are not limited to any particular receptacle or source geometry.

As noted previously, the radionuclide source receptacle 110, and the radionuclide source 104 disposed therein, may be moved between an extended and retracted configuration. In the extended configuration the radionuclide source receptacle 110 may be exposed to an exterior environment surrounding the holder 100. Correspondingly, when the radionuclide source holder 100 is in the retracted configuration, the radionuclide source receptacle may be retained within an interior of the radionuclide source holder 100 and optionally isolated from the exterior environment. For example, the radionuclide source receptacle 110 and the source 104 disposed therein may extend out from a housing, such as the distal end portion of the depicted tube, of the holder 100 in the extended configuration. In one such embodiment, the radionuclide source holder 100 may include an elongated housing 112, such as the depicted tube or other structure. The elongated housing 112 may include a channel that extends at least partially, and in some instances completely through the elongated housing. A rod 106 may extend at least partially through the depicted channel of the housing 112 and a distal portion of the rod, or other appropriate linearly translatable component, may be attached to the radionuclide source receptacle 110. Optionally a handle 102, or other structure configured to be grasped or driven in a desired direction, may be connected to a proximal portion of the depicted rod to apply forces to the rod oriented in a direction parallel to the longitudinal axis of the rod to axially move the radionuclide source receptacle 110 between the extended and retracted configurations due to corresponding axial movement of the rod. In the retracted configuration, the radionuclide source receptacle 110 may be retracted into an interior of the holder 100 or otherwise moved to a configuration where it is isolated from an exterior surrounding the holder 100. For instance, in the depicted embodiment shown in FIGS. 3A and 3B, the radionuclide source receptacle 110 is retracted into a distal end portion of the holder when it is in the retracted configuration. Similarly, in the extended configuration, the rod 106 displaces the radionuclide source receptacle 110 out of a distal opening of the housing 112 such that the radionuclide source 104 extends distally from the distal opening of the housing 112.

As noted above, in the retracted configuration shown in FIG. 3B, the precursor radionuclide source 104 may be isolated from a surrounding ambient environment, which includes a potentially associated container positioned in a radionuclide generator. In some embodiments, when the source 104 is isolated from the surrounding environment (e.g., is in the retracted configuration), it may be desirable to prevent leakage of the gaseous progeny radionuclides from the holder 100. Accordingly, in some embodiments, a radionuclide source holder 100 may include at least one seal 108 configured to form a gas-tight interface to isolate the radionuclide source 104 from the surrounding environment when the holder 100 is in the retracted configuration. For example, a radionuclide source holder may include a plurality of seals, each forming a gas-tight interface between the radionuclide receptacle 110, the rod 106, and/or any other appropriate portion of the holder 100 with an adjacent portion of the housing 112. For example, one or more seals 108 may be disposed on the radionuclide source receptacle 110 at a location either distally and/or proximally from a portion of the radionuclide source receptacle 110 in which the radionuclide source 104 is disposed. For example, in the depicted embodiment, a single O-ring is disposed on a proximal side of the source 104 to form a gas-tight interface with an internal surface of the housing 112. The depicted embodiment also includes one or more O-rings 108 (e.g., two O-rings as shown in the figure) disposed distally from the source 104 to form a gas-tight seal between the depicted O-rings and the internal surface of the housing 112 when the holder 100 is in the retracted configuration with the illustrated seals 108 in contact with an adjacent portion of the housing 112. It will be appreciated that any number of O-rings or other types of seals may be included at any appropriate location in a radionuclide source holder according to the present disclosure.

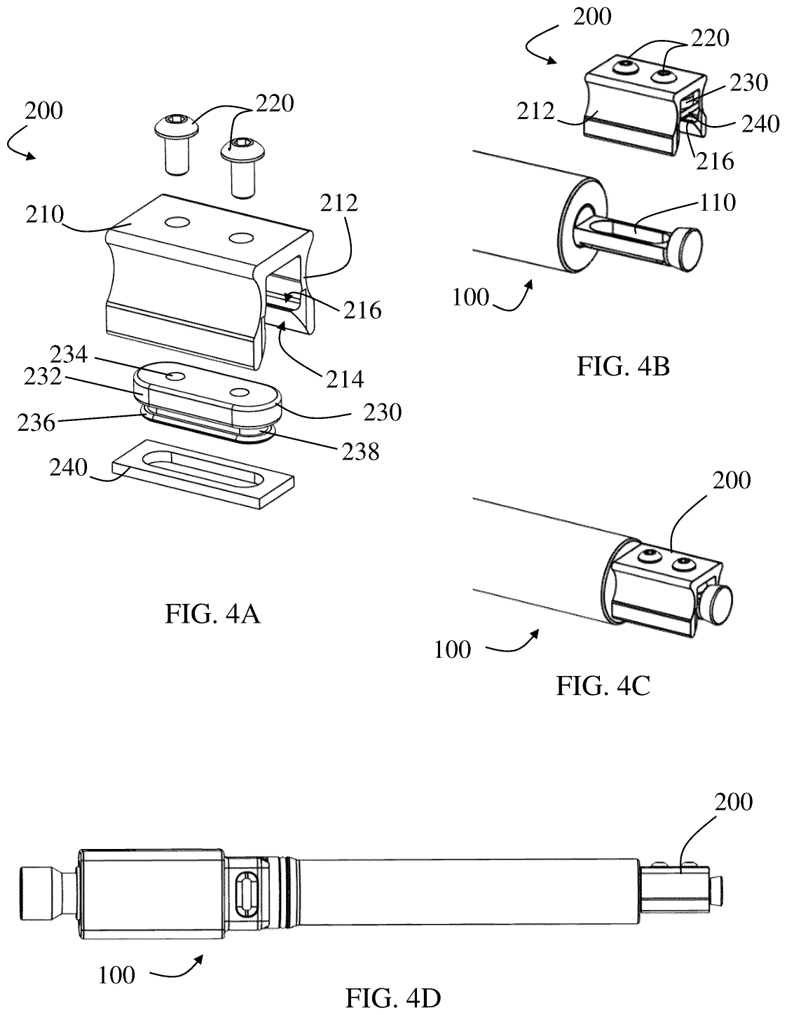

FIG. 4A shows a radionuclide source clip, while FIGS. 4B-4D show various views of a radionuclide source clip 200 relative to an extended distal end portion of a radionuclide source holder 100. As shown in the exploded view of the radionuclide source clip 200 in FIG. 4A, the radionuclide source clip 200 may include a body 210, a portion of one or more detents 220, a gasket holder 230, and a gasket 240. In some embodiments, the portions of the detents 220 located on the clip 200 may correspond to threaded fasteners, rivets, spring biased blungers, depressions, and/or any other structures that may function as part of a detent connection to provide a desired retaining force to the clip during operation. Each component of the radionuclide source clip, in some embodiments, may independently be made of materials that are resistant to degradation from exposure to radiation. For instance, each component of the radionuclide source clip may independently be made of metal, plastic, natural or synthetic polymer, or any other appropriate material. In some embodiments, some or all of the components of the radionuclide source clip may include materials that shield radiation.

It should be understood that a radionuclide source clip 200 may be any of a variety of shapes and sizes depending on the construction of the corresponding extendable portion of a radionuclide source holder 100. Regardless the radionuclide source clip 200 may be configured to be attached to the extended portion of the radionuclide source receptacle 110 of the holder 100 to maintain the radionuclide source therein when the clip 200 is attached. Thus, in some embodiments, the radionuclide source clip 200 is sized and shaped to engage with a portion of the radionuclide source holder 100 such as the depicted radionuclide source receptacle 110 extending out from a housing of the holder 100. According to some embodiments, the shape of the radionuclide source clip 200 may be sized and shaped to compliment a corresponding portion of the radionuclide source holder 100 to facilitate engagement therebetween while maintaining the source disposed in the radionuclide source receptacle 110 as elaborated on further below. For example, in some embodiments, the radionuclide source clip 200 may be sized and shaped to selectively attach to a portion of the radionuclide source receptacle 110 when it is in the extended configuration and extends distally from a distal end portion of the radionuclide source holder 100.

FIG. 4A shows the body 210 of the radionuclide source clip 200. A pair of opposing legs 212 disposed on either side of a channel extending along a longitudinal length of the clip body 210 may extend away from the clip body 210. The legs 212 may be configured to be engaged with and retain the clip 200 on a portion of the radionuclide source holder 100 such as the radionuclide source receptacle. For example, each of the legs 212 may include a latch including an angled surface 214 and a shelf 216 disposed on a proximal side of the angled surfaces. Thus, the legs 212 may be configured to slide over and past the associated portion of the radionuclide source holder 100 to place the shelves 216 in contact with an associated supporting surface of radionuclide source holder 100 to prevent relative movement of the clip 200 and the radionuclide source holder 100. For example, when the corresponding portion of the radionuclide source holder 100 is inserted between the legs 212 of the radionuclide source clip 200, the radionuclide source holder 100 presses against the angled surfaces 214 of the legs 212 to bias the legs 212 outwards to allow the radionuclide source holder 100 to move past the angled surfaces 214 and the shelf 216 such that the radionuclide source holder is pressed and held against the gasket of the radionuclide source clip (as shown in FIG. 4C). In such a position, the shelf 216 of each leg 212 of the radionuclide source clip are positioned to retain the clip 200 on the extended distal end portion of the radionuclide source holder 100 which may correspond to the radionuclide source receptacle 110 as elaborated on further below.

As also shown in the figures, the gasket holder 230 may be attached to an internal portion of the clip, for example, between the opposing legs 212 that extend away from body 210 of the clip 200. The gasket holder 230 may be retained in the channel of the clip 200 at a location between the two legs 212 of the body 210 using any appropriate type of attachment including, for example, interlocking mechanical features, adhesives, welds, threaded fasteners (e.g., the illustrated screws that may function as detent 220 that are engaged with threaded holes 234 formed in a first portion 232 of the gasket holder 230), and/or any other appropriate type of connection to retain the gasket holder 230 in a desired location. The gasket holder 230 may include a second portion 234 that extends towards the radionuclide source holder when the clip 200 is disposed thereon. In some embodiments, the second portion 234 of the gasket holder 230, or other portion of the clip 200, may be configured to extend vertically below the gasket 240 relative to the clip body 210, or other portion of the clip 200, and into the radionuclide source holder receptacle when the clip 200 is attached thereto to maintain a desired position of the radionuclide source. Additionally, the gasket holder 230 may also be configured to retain the gasket 240 thereon using a groove 238 formed on and extending around the gasket holder 230. For example, in the figure the groove 238 is configured to have a size and shape corresponding to the size and shape of an opening formed in the gasket 240 to hold the gasket 240 on the gasket holder 230 at a desired location within the clip 200 between the legs 212 as elaborated on below. For example, the gasket 240 may be spaced apart from the shelves 216 of the legs 212 to permit the radionuclide source receptacle 110 to be compressed between the gasket 240 and the shelves 216 of the legs 212 of the clip.

It may be desirable to both maintain a position of a radionuclide source within the radionuclide source holder 100 as well as to seal the radionuclide source. Accordingly, in some embodiments, the depicted gasket 240 may be made from a compliant material such that when the gasket 240 is pressed against a surface surrounding the radionuclide source receptacle 110 when the clip 200 is attached to the radionuclide source holder 100, the gasket may form a seal that isolates the radionuclide source from the ambient environment surrounding the holder 100 and clip 200. For example, the gasket 240 may be compressed between the radionuclide source receptacle 110 and the clip body 210. In either case, the gasket 240 and the gasket holder 230 of the radionuclide source holder 100 may facilitate forming a sealed volume with the radionuclide source receptacle 110 in which the radionuclide source 104 may be contained. As noted previously, a source 104 disposed in the radionuclide source receptacle 110 may emit gas. Accordingly, in some embodiments, the gasket 240 may be configured to form a gas-tight sealed volume. Thus, the formation of the sealed volume in which the radionuclide source may be maintained may desirably prevent the emission of gaseous radionuclides during handling and transport of the radionuclide source holder 100. It should be understood that the depicted gasket 240 may be made from any appropriate deformable and/or elastically deformable material capable of creating the desired seal as the disclosure is not so limited. Note that, in some embodiments, when the radionuclide source clip is engaged with the radionuclide source holder, there may not be a gas-tight sealed volume.

In addition to the above, in some embodiments, a portion of the gasket holder 230 or other portion of the clip 200 may at least cover, and in some embodiments, extend partially into the radionuclide source receptacle 110 (e.g., the second portion 236 of the gasket holder 230) to maintain a desired position of the radionuclide source within the radionuclide source receptacle 110 when the clip 200 is attached to the holder 100, e.g., in an engaged configuration. For example, FIGS. 4B and 4C show a perspective view of the radionuclide source clip 200 engaging with a distal end portion of radionuclide source holder 100. The legs 212 of the radionuclide source clip 200 are configured to engage with and maintain the clip 200 on the distal end portion of the radionuclide source holder 100 and to compress the gasket 240 against the radionuclide source receptacle 110 when the clip 200 is positioned thereon. Accordingly, in some such embodiments when a radionuclide source is present in the receptacle, the attachment of the radionuclide source clip 200 to the radionuclide source holder 100 may facilitate the formation of a sealed volume and/or may maintain a location of a radionuclide source within the radionuclide source receptacle 110. However, it should be understood that in some embodiments, while the radionuclide source clip does not form a sealed volume, it may still physically maintain a position of the radionuclide source material.

While the embodiment depicted in FIGS. 4A-4D show the radionuclide source clip 200 includes two legs 212 with angled clips configured to engage with the radionuclide source holder 100, it should be understood that any number of legs and/or other configurations of a radionuclide source clip 200 configured to be engaged with a radionuclide source holder 100 may be used as the disclosure is not so limited. For example, the radionuclide source clip 200 may be configured to be connected to the radionuclide source holder 100 through any of a variety of connectors. In some embodiments, the radionuclide source clip may engage with the radionuclide source holder via one or more detents, pin-in-hole connections, hook-and-loop connectors, interlocking mechanical features, a magnetic connector, and/or any other appropriate type of connector capable of selectively connecting the radionuclide source clip 200 to the corresponding portion of the radionuclide source holder 100.

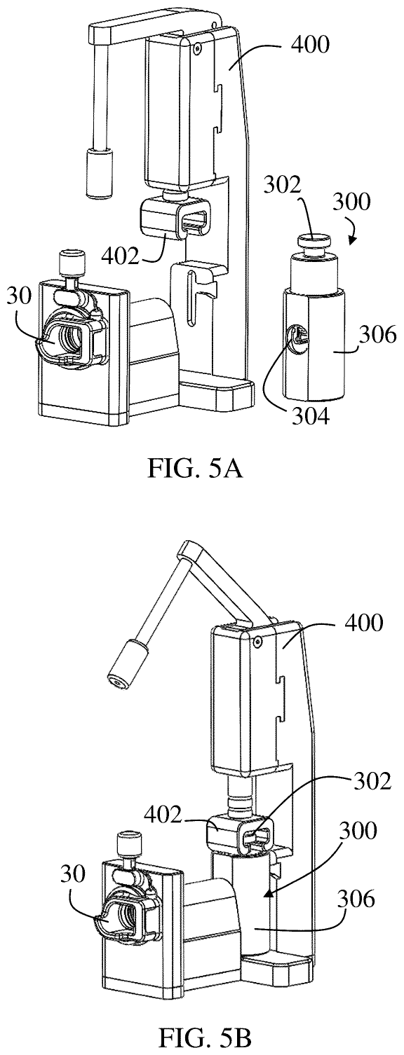

FIGS. 5A-5B show different schematic diagrams of some or all of a system for removing a clip from a radionuclide source holder as described herein. FIG. 5A is a perspective view of an example system for removing a radionuclide source clip 200 from a radionuclide source holder 100. The system includes a radionuclide source clip handling system 300, a manipulator 400, and a support 30. The radionuclide source clip handling system 300 is configured to be engaged with the manipulator 400 or other appropriate type of actuator where the manipulator 400 may be configured to actuate the radionuclide source clip handling system 300 between two or more configurations during removal of a clip from a radionuclide source holder 100 as elaborated on further below. For example, a plunger 302 of the radionuclide source clip handling system 300 may be configured to be engaged within an end effector 402, or other appropriate attachment, of the manipulator 400. For example, mechanically interlocking features of the plunger 302 and end effector may be engaged to permit movement of the end effector 402 of the manipulator 400, or other actuator, to move the plunger 302 in a desired direction. For example, the manipulator 400 may be configured to move the end effector 402 in a vertical direction relative to an underlying supporting surface when a handle, or other trigger, of the manipulator 400 is actuated to displace the connected portion of the plunger 302, see the initial unactuated configuration shown in FIG. 5A and an actuated configuration of the system in FIG. 5B. This may facilitate the automation and/or manipulation of the system in applications where the system is used in an isolated environment.

During removal of a clip, it may be desirable to hold a radionuclide source holder 100, in a desired pose (i.e., position and orientation) relative to a radionuclide source clip handling system 300. Thus, a support 30 may be configured to receive and support at least a portion of a radionuclide source holder, e.g., radionuclide source holder 100 as shown in FIG. 2, in a predetermined pose relative to the radionuclide source clip handling system 300 and/or manipulator 400. In some embodiments, the support 30 may be attached to, or otherwise fixed relative to a position, of the manipulator 400. For example, the support 30 may be used to align a distal end portion of the radionuclide source holder 100 with an opening 304 of an outer housing 306 of the radionuclide source clip handling system 300 when the radionuclide source clip handling system 300 is engaged with the manipulator 400 or other appropriate portion of a system. In some embodiments, the support 30 may include a lock configured to maintain the radionuclide source holder 100 in a desired location and orientation relative to the radionuclide source clip handling system 300 during removal of a radionuclide source clip 200 from the radionuclide source holder 100.

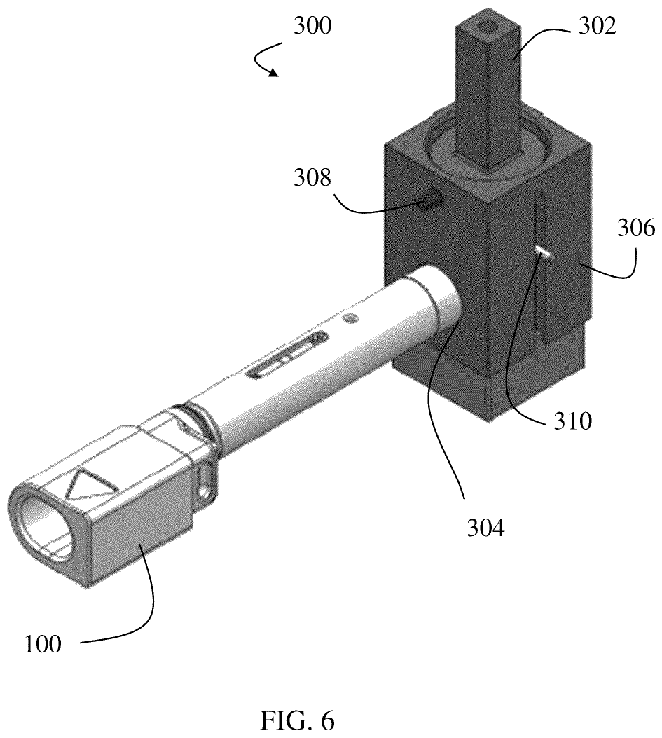

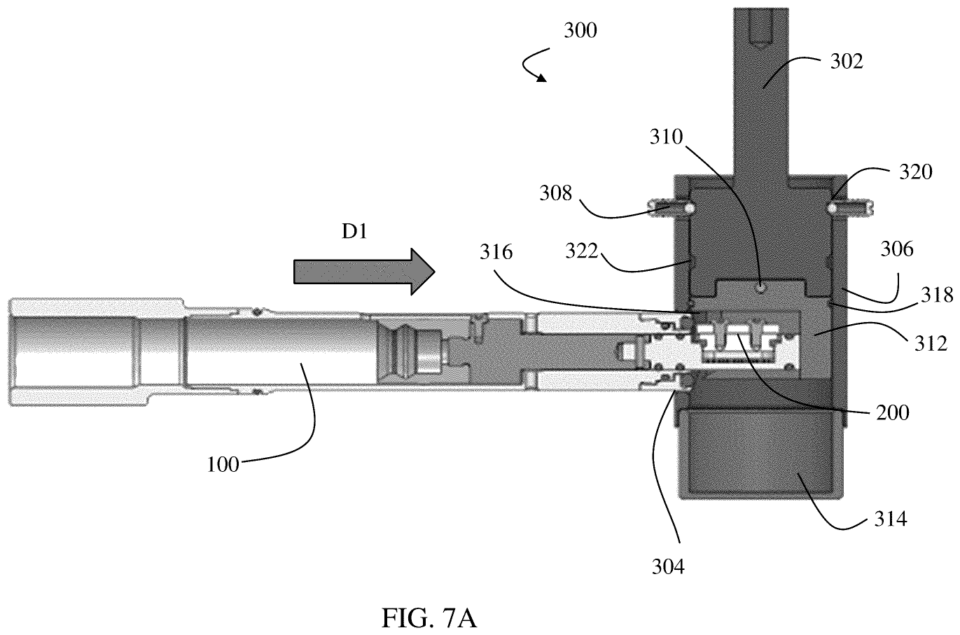

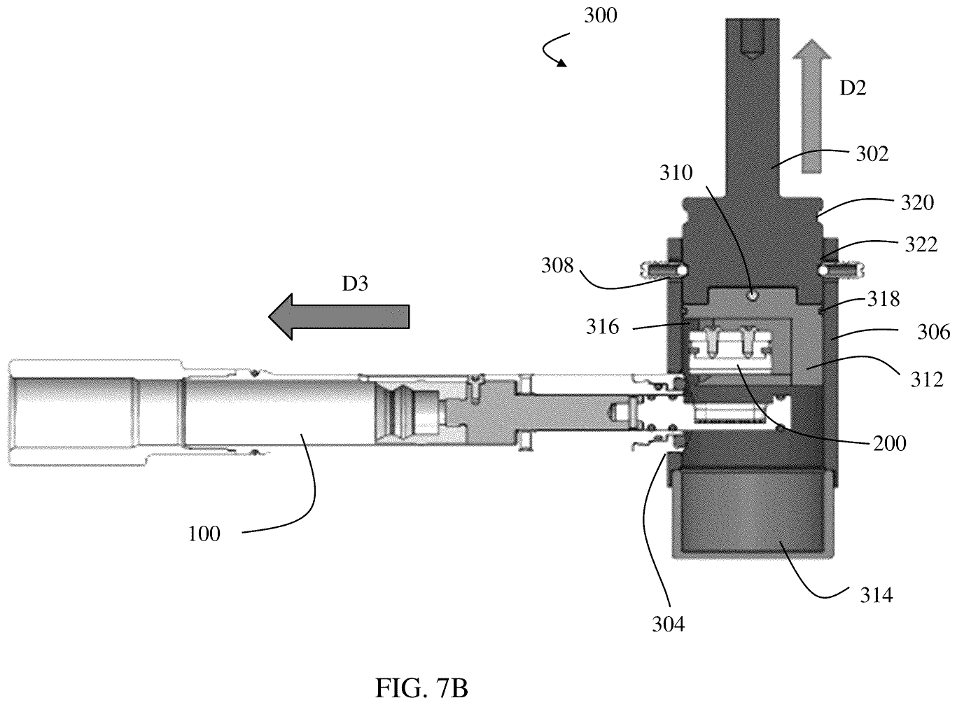

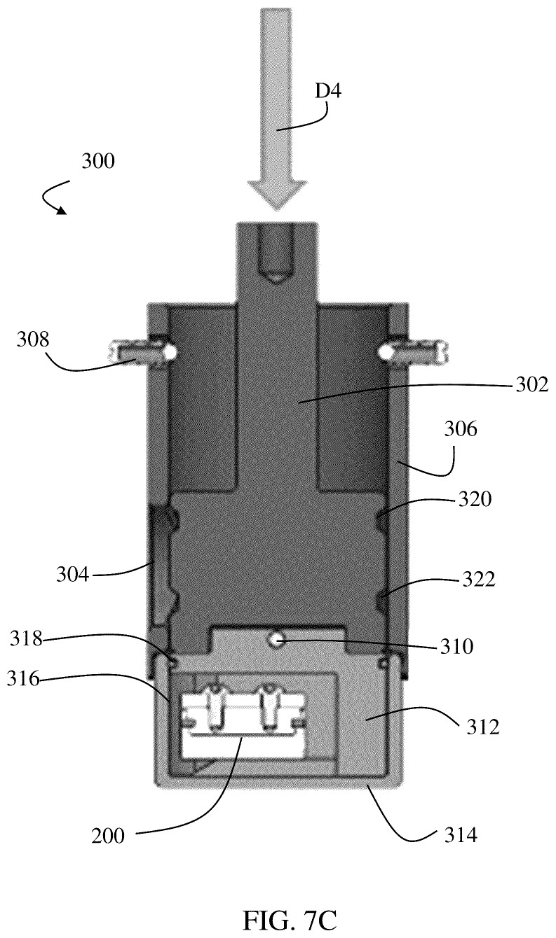

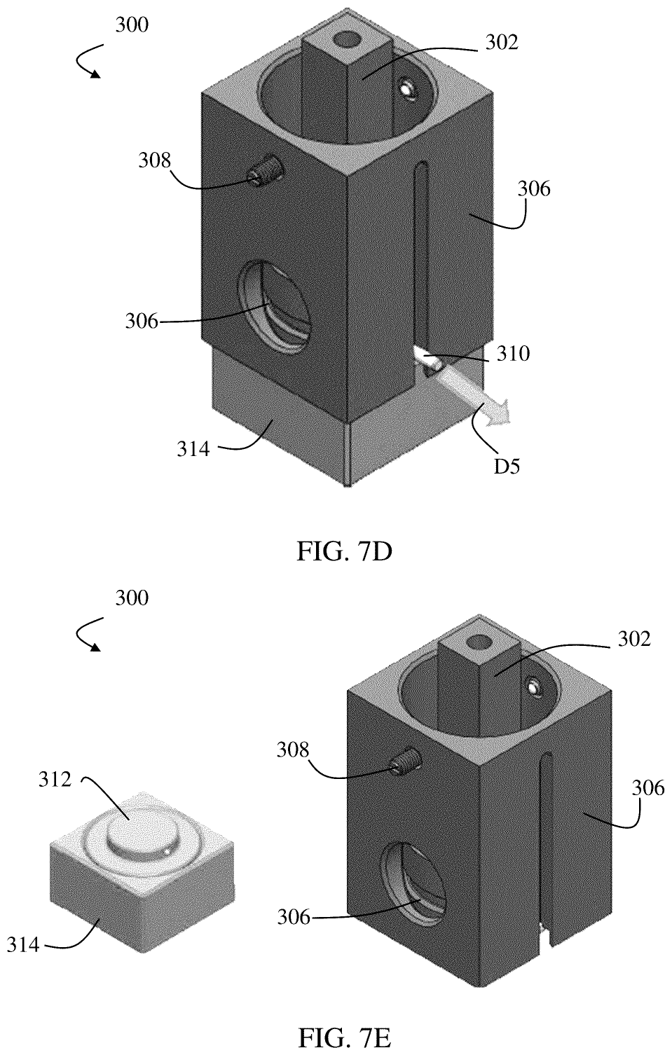

FIG. 6 is a perspective view of a radionuclide source holder 100 where a distal end portion of the radionuclide source holder 100 is extending through an opening 304 (e.g., a second opening) formed into an outer housing 306 of a radionuclide source clip handling system 300. This view shows that the outer housing 306 may include a first set of one or more detents 308 configured to maintain an initial configuration of the system prior to actuation. For example, the one or more detents 308 may be configured to maintain an initial position of a plunger 302, a first clip housing portion 312 and second clip housing portion 314, as shown in FIGS. 7A-7C, which are described in more detail below. Referring again to FIG. 6, the outer housing 306 includes a lock 310 that attaches a first clip housing portion 312 to the plunger 302 within the outer housing 306 which, for example, may maintain a location of the first clip housing portion 312 relative to the plunger 302 such that the first clip housing portion 312 may move in sync with the plunger 302 or other appropriate actuatable portion of the clip handling system. The lock may be moved from a locked to an unlocked configuration to release the first clip housing portion 312 from the plunger 302, as described in more detail below. The lock 310 may be any of a variety of locks, such as the depicted locking pin and hole, magnets, interlocking mechanical features, latches, or any other type of lock that can be moved from a locked configuration to an unlocked configuration to selectively retain or release the first clip housing portion 312 on the plunger 302. While not shown for the sake of clarity in this figure, as previously shown in FIGS. 5A and 5B, a support 30 may also be present to support and/or align a distal end portion of the radionuclide source holder 100 with the opening 304 when inserting the distal end portion of the holder 100 into the outer housing 306.

FIGS. 7A-7C show cross-sectional views of one embodiment of a radionuclide source clip handling system 300 in various configurations with and without the radionuclide source holder 100 inserted therein. FIG. 7A depicts the radionuclide source clip handling system 300 with a distal end portion of the radionuclide source holder 100 inserted into the system 300. As noted above, and as can be seen in the cross-sectional view, the outer housing 306 has an internal volume containing a first clip housing portion 312 and second clip housing portion 314. Additionally, a plunger 302, optionally disposed against and selectively attached to the first clip housing portion 312 by lock 310 as described above, may at least extend partially into the internal volume of the outer housing 306. The plunger 302 and the first clip housing portion may be disposed within a first portion of the internal volume defined by the outer housing 306. Additionally, both of the first clip housing portion 312 and the plunger 302 may be configured to linearly translate through at least a portion of the internal volume of the outer housing 306. The second clip housing portion may be disposed within a second portion of the internal volume defined by the outer housing 306, opposite the first portion, such that the linear translation of the first clip housing portion 312 and the plunger 302 may be towards and/or away from the second clip housing portion 314 depending on the direction of actuation. As will be appreciated, each of the first and second clip housing portions 312 and 314 may occupy a portion of the internal volume of the outer housing 306 and may further independently define a portion of the internal volume, e.g., a portion that may be configured to receive a distal end portion of a radionuclide source holder 100. Additionally, as illustrated in the figures, in some embodiments, the second clip housing portion may extend partially out of the internal volume of the outer housing 306. However, embodiments in which both clip housing portions are fully disposed within the outer housing 306 are also contemplated.

The first clip housing portion 312 includes an opening 316 aligned with the opening 304 formed in the outer housing 306 and through which the distal end portion of the radionuclide source holder 100 may be inserted when the first clip housing portion is in the initial configuration. That is, the distal end portion of the radionuclide source holder 100 is inserted through opening 304 of the outer housing 306 and the opening 316 of the first clip housing portion 312. The first clip housing portion may also include one or more seals 318 extending around a perimeter of the first clip housing portion to form a sliding seal with the adjacent internal surface of the outer housing and/or to form a sealed volume with the second clip housing portion 314 when in the sealed configuration, as described in the context of FIG. 7C.

Referring again to FIG. 7A, the first clip housing portion 312 of the system 300 is in an initial configuration when the radionuclide source holder 100 with the radionuclide source clip 200 disposed on the distal end portion is initially inserted into the opening 304 of the outer housing 306 and the opening 316 of the first clip housing portion 312. Additionally, the plunger 302, or other appropriate portion of the system, may include one or more grooves 320 and 322, indents, magnets, protrusion, latches, or other features configured to be selectively engaged with the one or more detents 308 associated with the outer housing 306. The portions of the detents formed on the plunger 302 and the outer housing 306 may be configured to maintain the plunger 302 and the associated first clip housing portion 312 in the one or more desired configurations until an appropriate force is applied to move the system to another configuration. For example, the depicted first groove 320 and second groove 322 at a second location on the plunger 302 may be located to facilitate selective engagement (e.g., locking) of the set of one or more detents 308 and the plunger 302 at an initial configuration and a retracted configuration as detailed further below.

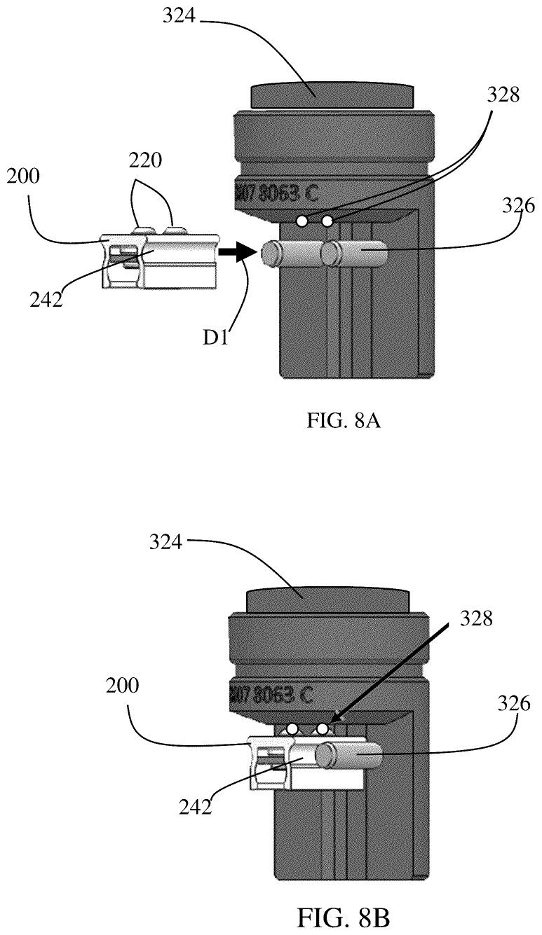

Referring again to FIG. 7A, upon insertion of the radionuclide source holder 100 in direction D1 into the opening 304 of the outer housing 306 and the opening 316 of the first clip housing portion 312, a radionuclide source clip interface 324 of the first clip housing portion 312 selectively engages with the radionuclide source clip 200 as the radionuclide source holder 100 is inserted. The radionuclide source clip interface 324 may be configured to apply forces to the radionuclide source clip 200 at least in a direction that is at least partially perpendicular, and in some instances substantially perpendicular, to a direction of insertion of the radionuclide source holder 100 into the outer housing 306. The radionuclide source clip interface 324 can be seen in more detail in the context of FIGS. 8A and 8B, which show cutaway views of the radionuclide source clip handling system 300 that depict the first clip housing portion 312 and the radionuclide source clip 200. The first clip housing portion 312 includes a radionuclide source clip interface 324 which is configured to support the radionuclide source clip 200. For example, radionuclide source clip 200 is configured to slide in the direction of arrow D1 as shown in FIG. 8A such that the clip 200 is selectively engaged with the radionuclide source clip interface 324 as shown in FIG. 8B. The radionuclide source clip interface 324 may be configured to engage with the radionuclide source clip 200 disposed on a radionuclide source holder 100, as shown in FIG. 7A, when the radionuclide source holder 100 and associated radionuclide source clip 200 is disposed in an opening (e.g., a first opening 316) of the first clip housing portion 312. Additionally, it will further be appreciated that FIGS. 8A and 8B show the interaction between the radionuclide source clip 200 and the radionuclide source clip interface 324 in the absence of the radionuclide source holder 100 for clarity, but the depicted engagement between the clip 200 and radionuclide source clip interface 324 and the holder 100 depicted in these figures may be used with any of the embodiments disclosed herein.

In some embodiments, and as shown in FIG. 8B, the radionuclide source clip interface 324 may include one or more rails 326 configured to slidingly engage with one or more grooves 242 formed on and extending along a length of the legs of the radionuclide source clip 200, along which the clip 200 may be supported and/or may slide. Other supporting structures and/or connectors are also possible, and may be selected to selectively engage with features present on the clip 200 to selectively retain the clip 200 on clip interface 324 relative to a direction in which the clip interface and associated portion of the first clip housing portion 312 associated with and partially surrounding the clip interface 324. Additionally, the radionuclide source clip interface 324 may include a one or more detents 328 (e.g., illustrated as ball plungers in the figure) present on the first clip housing portion 312 that interfaces with a portion of one or more corresponding portions of the detents 220 formed on the clip 200 as shown FIG. 8B. The one or more detents 328 of the radionuclide source clip interface 324 may be configured to selectively engage with the radionuclide source clip 200 when the distal end portion of the radionuclide source holder 100 is positioned within the opening and apply a retaining force to the clip to resist removal of the clip 200 from the depicted rails 326 or other supporting structure.

As noted above, the rails 326 (or other supporting structure and/or connector) of the radionuclide source clip interface 324 and/or the one or more detents 328 of the first clip housing portion 312 may maintain a position of the radionuclide source clip within the interior volume of the housing (e.g., a first clip housing portion 312) of the radionuclide source clip handling system 300. For example, the one or more detents 328 on the first clip housing portion 312, when engaged with the portion of one or more detents of 220 on the clip 200, may prevent movement of the clip 200 that is parallel to the rails or other supporting features of the radionuclide source clip interface 324 unless a force that is larger than a threshold force is used to overcome the engagement between the one or more detents 328 and the portion of one or more detents of 220 on the clip 200. The one or more detents 328 of the first clip housing portion 312 may be any of a variety of appropriate detent of connection to prevent lateral movement of the clip 200. Additionally, when engaged with the radionuclide source clip interface, movement of the first clip housing portion 312 due to movement of the associated plunger 302, or other structure, may apply a force to the radionuclide source clip that is perpendicular to the direction in which the clip may slide along the rails 326 or other supporting structure and/or connector.

In some embodiments, in an initial configuration of the radionuclide clip interface 324 and first clip housing portion 312 may be aligned with an opening 304 of the outer housing 306 such that upon insertion of a distal end portion of a radionuclide source holder 100 with a radionuclide source clip 200 disposed thereon, the radionuclide source clip 200 may be engaged with the radionuclide source clip interface 324. As shown in FIG. 7B, after the radionuclide source clip 200 selectively engages with the radionuclide source clip interface 324, the plunger 302 may be moved in direction D2 to move the first clip housing portion 312, radionuclide source clip interface 324, and the attached radionuclide source clip 200 away from the holder 100 and optionally the second clip housing portion 314 to a retracted configuration. For example, the legs of the clip 200 may be deformed over and past the associated portion of the holder 100 to disengage the clip 200 from the holder when the plunger 302 is moved in the indicated direction. In some embodiments, the set of one or more detents 308 engaged with one or more first grooves 320 are disengaged and moved into engagement with a one or more second grooves 322 when a sufficient force to overcome the engagement between the set of one or more detents 308 and the one or more first grooves 320 is applied to the plunger 302 to move the plunger 302 to the retracted configuration.

This corresponding movement of the first clip housing portion 312 and the radionuclide source clip interface 324 from the initial configuration to the retracted configuration may remove the radionuclide source clip 200 from the radionuclide source holder 100 due to the radionuclide source holder 100 being held stationary in the opening 304 of the outer housing 306 by the associated supports configured to maintain a desired pose of the radionuclide source holder 100 and the radionuclide source clip handling system 300 during use. Additionally, the movement of the first clip housing portion 312 and the radionuclide source clip interface 324 from the initial configuration to the retracted configuration may take the opening 316 of the first clip housing portion 312 out of alignment with the second opening 304 of the outer housing 306. In either case, the radionuclide source interface 324 may remain engaged with the radionuclide source clip 200 such that the clip 200 is now spaced from the radionuclide source holder 100. Additionally, the opening 316 of the first clip housing portion 312 is no longer aligned with the opening 304 of the outer housing 306. Following removal of the radionuclide source clip 200, the radionuclide source in the receptacle of the radionuclide source holder 100 is exposed and the radionuclide source holder 100 may be changed from an extended configuration to a retracted configuration as shown in FIGS. 3A and 3B. The radionuclide source holder 100 may then be removed in direction D3 which may be at least partially perpendicular, and in some embodiments substantially perpendicular, to a direction of motion of the plunger 302 and radionuclide source clip interface 324 to remove the holder 100 from the through the radionuclide source clip handling system 300.

FIG. 7C shows that, after removing the radionuclide source holder from the housing, the plunger 302 may then be depressed in direction D4 to move the first clip housing portion 312 from the retracted configuration towards the second clip housing portion 314 to the sealed configuration. In some embodiments, this displacement may be continued until the first clip housing portion 312 is engaged with and forms a sealed volume with the second clip housing portion 314. For example, in the depicted embodiment, the first clip housing portion 312 is at least partially inserted into the second clip housing portion such that the first opening 316 of the first clip housing portion 312 is displaced into and covered by an interior surface of the second clip housing portion 314. As shown in the figure, the radionuclide source clip 200 and associated radionuclide source clip interface 324 may be retained by the first clip housing portion 312 and thus may be positioned within the interior volume formed between the first and second clip housing portions 312 and 314 when the first clip housing portion 312 is moved to the sealed configuration in engagement with the second clip housing portion 314.

The use of the one or more seals 318 to form a sealed volume between the first and second clip housing portions 312 and 314 may be desirable, for example, for inhibiting the emanation of any radionuclides from the housing portions when in the sealed configuration and forming a sealed volume. For example, there may be a trace amount of radionuclides present on the radionuclide source clip 200 after it was engaged with the radionuclide source holder 100 and/or on the interior surfaces of the first and/or second clip housing portions 312 and 314 after the radionuclide source in the radionuclide source holder 100 was exposed following removal of the clip 200. Thus, it will also be appreciated that the one or more seals 318 may be configured to form a seal between adjacent surfaces of the first and second housing portion 312 and 314 when in the sealed configuration. For example, a cross sectional shape of an external surface of the first clip housing portion may conform to a cross sectional shape of the internal volume such that the seal 318 may be compressed between adjacent portions of the first and second clip housing portions 312 and 314 to form a sealed volume with the clip 200 disposed therein. In addition to the above, in some embodiments, the first and second clip housing portions 312 and 314 may be constructed from radiation shielding materials, as described elsewhere herein. Note that in some embodiments the housing portions 312 and 314 may simply be constructed from radiation resistant material rather than shielding materials.

FIGS. 7D and 7E show a perspective view of the radionuclide source clip handling system 300 when the system is in the sealed configuration. Following movement of the plunger 302 such that the first clip housing portion 312 is engaged with and optionally sealed by the second clip housing portion 314, the lock 310 may be disengaged to release the sealed first and second clip housing portions 312 and 314 from the radionuclide source clip handling system 300. In this embodiment, the lock 310 includes a pin-in-hole mechanism where the pin is removed in direction D5 to release the sealed first and second clip housing portions 312 and 314 from the plunger 302. Note that the lock 310 may include any of a variety of locks, as described above. Once released, the sealed first and second clip housing portions 312 and 314 containing the radionuclide source clip 200 may be removed from the outer housing 306 and disposed of if desired. In some embodiments, a second set of first and second clip housing portions 312 and 314 may then be attached to the radionuclide source clip handling system 300 for another use.

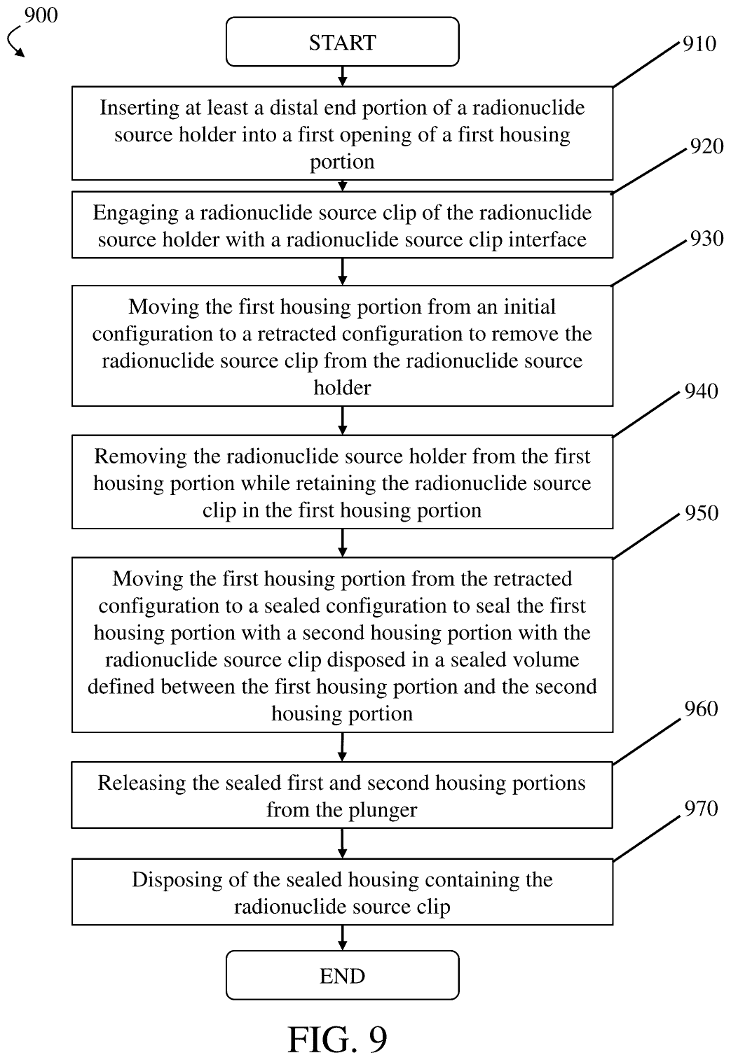

FIG. 9 is method flow diagram showing how to the use the systems described herein, in accordance with some embodiments. The method 900 includes inserting at least a distal end portion of a radionuclide source holder into a first opening of a first clip housing portion 910. The radionuclide source holder 100 may be inserted into the radionuclide source clip handling system 300 in a direction D1 as shown in FIG. 7A that is perpendicular to a direction D4 in which the plunger 302 may linearly translate. Inserting the radionuclide source holder 100 may facilitate the sealing of the interior volume of the housing (e.g., the outer housing 306 and/or the first clip housing portion 312) by one or more seals that are present on the exterior of the radionuclide source holder 100, as shown as elements 152 in FIGS. 3A and 3B.

When inserting the radionuclide source holder 100, the first opening 316 of first clip housing portion 312 may be in an initial configuration where an opening of the first clip housing portion 312 is aligned with the opening 304 of the outer housing 306, e.g., to facilitate insertion of the distal end portion of the radionuclide source holder 100. The distal end portion of the radionuclide source holder 100 includes a receptacle 162 in which a radionuclide source 132 may be contained, in accordance with some embodiments. When inserting the radionuclide source holder 100, a radionuclide source clip 200 of the radionuclide source holder 100 may be engaged with the distal end portion of the radionuclide source holder 100, for example, over the receptacle 110 to maintain a position of the radionuclide source 132 in the receptacle 162, as shown in FIG. 4C.

The method 900 may include engaging a radionuclide source clip of the radionuclide source holder with a radionuclide source clip interface at 920. Note that the radionuclide source clip interface 324 may be connected to the first clip housing portion 312. The first clip housing portion 312 may be in an initial configuration such that the opening 316 of the first clip housing portion 312 is aligned with the second opening 304 of the outer housing 306, e.g., as shown in FIG. 7A, to facilitate the engaging of the radionuclide source clip 200 with the radionuclide source clip interface 525. Engagement of the radionuclide source clip 200 with the radionuclide source clip interface 525 may proceed as shown and described in the context of FIGS. 8A and 8B. Engaging the radionuclide source clip 200 with the radionuclide source clip interface 324 may proceed by selectively attaching the clip 200 to the interface 324, e.g., by sliding the clip 200 along rails or other support of the interface 324. Other methods of engagement, e.g., using magnets, interlocking grooves, or the like are also possible.

The method 900 may further include moving the first clip housing portion from an initial configuration to a retracted configuration to remove the radionuclide source clip from the radionuclide source holder at 930. Moving the first clip housing portion 312 from an initial configuration to a retracted configuration may proceed by linearly translating a plunger 302 which may be engaged with the first clip housing portion 312, e.g., in a direction D2 as shown in

FIG. 7B. This movement facilitates removal and spacing of the radionuclide source clip 200 from the radionuclide source holder 100 as further shown in FIG. 7B because the radionuclide source clip interface 324 engaged with the radionuclide source clip 200 is connected to the first clip housing portion 312. FIG. 7B also shows that, in some embodiments, the movement from the initial configuration to the retracted configuration may be a predetermined amount of movement. For instance, the distance may be determined by the interaction between the one or more detents 308 and the one or more grooves 320 and 322 in plunger 302.

Removing the radionuclide source clip 200 may expose a radionuclide source 104 of the radionuclide source holder 100. Accordingly, in some embodiments, the radionuclide source holder 100 may then be changed from an extended configuration as shown in FIG. 3A and 7B to a retracted configuration as shown in FIG. 3B prior to removal of the radionuclide source holder 100 from the first clip housing portion 312 and overall system 300 while retaining the radionuclide source clip 200 in the first clip housing portion 312. Accordingly, the method 900 may include removing the radionuclide source holder from the first clip housing portion while retaining the radionuclide source clip in the first clip housing portion 940. The removing may proceed by linearly translating the radionuclide source holder 100 in direction D3 as shown in FIG. 7B. Upon removal the exposed radionuclide source 104 of the holder 100 may be used for any desired application, including, for example, usage with a radionuclide generator.

The method 900 may further include moving the first clip housing portion from the retracted configuration to a sealed configuration to seal the first clip housing portion with a second clip housing portion with the radionuclide source clip disposed in a sealed volume defined between the first clip housing portion and the second clip housing portion at 950. This may include linear translation of the associated plunger 302 until the first opening 316 of the first clip housing portion 312 is displaced into and covered by an interior surface of the second clip housing portion 314 as described previously relative to FIG. 7C. When moving the first clip housing portion 312, the radionuclide source clip 200 may remain engaged with the radionuclide source clip interface 324 of the first clip housing portion 312, and thus the radionuclide source clip 200 may be sealed within the interior volume defined by the first and second clip housing portions 312 and 314 in the sealed configuration. FIG. 7C additionally shows that sealing of the first and second clip housing portions 312 and 314 may, for example, be provided by one or more seals 318 associated with the first clip housing portion 312. The one or more seals 318 may include an o-ring, an elastomer, a gasket, or any other suitable seal for forming a gas-tight seal.

According to some embodiments, the method 900 includes releasing the sealed first and second clip housing portions from the plunger at 960. This may include disengaging a lock 310 as shown in FIGS. 7D and 7E. Following release of the sealed first and second clip housing portions 312 and 314, the method 900 may further include disposing or otherwise handling the sealed clip housing containing the radionuclide source clip at 970.

It should be understood that the method 900 may include some and/or all of the steps shown in method flow diagram of FIG. 9. In some embodiments, some and/or all of the steps may be repeated or performed simultaneously. Additionally, while the steps of the method shown in FIG. 9 are described in a sequential order, it is also possible that the steps may be performed in a different order than as illustrated in the method flow diagram and/or as described herein.

It will further be appreciated that while the above schematic diagrams and method steps are described in the context of removing a radionuclide source clip 200, the radionuclide source clip handling system 300 may also be configured and/or used to attach a radionuclide source clip 200 to a distal end portion of a radionuclide source holder 100, for instance, to cover and/or maintain a position of a radionuclide source therein. Attaching a radionuclide source clip 200 may proceed using the reverse of the above-described method steps. For instance, a method of attaching a radionuclide source clip 200 may include inserting a distal end portion of a radionuclide source holder 100 in a retracted configuration into the housing 306 of the radionuclide source clip handling system 10. The method may include changing a radionuclide source holder 100 from a retracted configuration to an extended configuration while the radionuclide source clip handling system 300 is in a misaligned configuration with the radionuclide source clip interface 324 out of alignment the opening 304 of the outer housing 306 (i.e., in a retracted configuration), as shown in FIG. 7B. The plunger 302 may then be moved from the retracted configuration towards the radionuclide source holder 100 to engage the radionuclide source clip 200 with the radionuclide source holder 100. This may be done, in some embodiments, by moving the plunger 302 in the opposite direction of D2 as shown in FIG. 7B to the configuration depicted in FIG. 7A. The method may then further include removing the radionuclide source holder 100 engaged with the radionuclide source clip 200 from the housing 306 of the handling system 300. In some embodiments, the radionuclide source holder 100 engaged with the radionuclide source clip 200 may then be inserted into a transport container, for instance, to transport the radionuclide source holder 100.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A radionuclide source clip handling system comprising:
a first clip housing portion;
a second clip housing portion, wherein the first clip housing portion is configured to be moved away from and towards the second clip housing portion;
a first opening formed in the first clip housing portion; and
a radionuclide source clip interface configured to retain a radionuclide source clip from a distal end portion of a radionuclide source holder positioned in the first opening, wherein the radionuclide source clip interface is connected to the first clip housing portion such that moving the first clip housing portion from an initial configuration to a retracted configuration is configured to remove the radionuclide source clip from the radionuclide source holder.

2. The radionuclide source clip handling system of claim 1, wherein the first clip housing portion is configured to be moved away from and towards the second clip housing portion between the initial configuration, the retracted configuration, and a sealed configuration.

3. The radionuclide source clip handling system of claim 2, wherein moving the first clip housing portion from the retracted configuration to the sealed configuration seals the first clip housing portion with the second clip housing portion.

4. The radionuclide source clip handling system of claim 2, wherein the second clip housing portion covers the first opening when the first clip housing portion is in the sealed configuration.

5. The radionuclide source clip handling system of claim 1, further comprising an outer housing including an internal volume, wherein the first clip housing portion is configured to move within the internal volume.

6. The radionuclide source clip handling system of claim 5, further comprising a second opening formed in the outer housing, wherein the second opening is aligned with the first opening when the first clip housing portion is in the initial configuration.

7. The radionuclide source clip handling system of claim 5, wherein the first clip housing portion is configured to linearly translate within the internal volume of the outer housing.

8. The radionuclide source clip handling system of claim 1, wherein the radionuclide source clip interface includes one or more rails configured to slidingly engage with one or more grooves formed on the radionuclide source clip.

9. The radionuclide source clip handling system of claim 1, wherein the radionuclide source clip interface includes one or more detents configured to selectively engage with the radionuclide source clip when the distal end portion of the radionuclide source holder is positioned within at least one of the first opening and a second opening.

10. The radionuclide source clip handling system of claim 9, wherein the one or more detents are configured to resist removing the radionuclide source clip from the radionuclide source clip interface.

11. The radionuclide source clip handling system of claim 1, further comprising a plunger configured to move the first clip housing portion between the initial configuration and the retracted configuration.

12. The radionuclide source clip handling system of claim 1, further comprising a support configured to support the radionuclide source holder in a predetermined pose.

13. The radionuclide source clip handling system of claim 1, further comprising the radionuclide source holder.

14. The radionuclide source clip handling system of claim 13, further comprising a radionuclide source disposed in a radionuclide source receptacle of the radionuclide source holder.

15. The radionuclide source clip handling system of claim 1, further comprising the radionuclide source clip.

16. A method of removing a radionuclide source clip, the method comprising:
inserting a distal end portion of a radionuclide source holder into a first opening of a first clip housing portion;
engaging the radionuclide source clip of the radionuclide source holder with a radionuclide source clip interface; and
moving the first clip housing portion from an initial configuration to a retracted configuration to remove the radionuclide source clip from the radionuclide source holder.

17. The method of claim 16, further comprising removing the radionuclide source holder while retaining the radionuclide source clip in the first clip housing portion in the retracted configuration.

18. The method of claim 17, wherein removing the radionuclide source clip from the radionuclide source holder exposes a radionuclide source of the radionuclide source holder.

19. The method of claim 17, further comprising moving the first clip housing portion from the retracted configuration to a sealed configuration to seal the first clip housing portion with a second clip housing portion with the radionuclide source clip disposed in a volume defined between the first clip housing portion and the second clip housing portion.

20. The method of claim 19, wherein the volume defined between the first clip housing portion and the second clip housing portion is a sealed volume.

21. The method of claim 16, further comprising inserting the distal end portion of the radionuclide source holder through a second opening formed in an outer housing that is aligned with the first opening of the first clip housing portion when the first clip housing portion is in the initial configuration.

22. The method of claim 21, wherein moving the first clip housing portion to the retracted configuration moves the first opening out of alignment with the second opening.

23. The method of claim 21, further comprising moving the first clip housing portion within an internal volume of the outer housing.

24. The method of claim 16, further comprising resisting removal of the radionuclide source clip from the radionuclide source clip interface.

25. The method of claim 16, further comprising using a plunger to move the first clip housing portion between the initial configuration and the retracted configuration.

26. The method of claim 25, further comprising releasing the first clip housing portion from the plunger.

27. The method of claim 16, further comprising linearly translating the first clip housing portion.

28. The method of claim 16, further comprising supporting the radionuclide source holder in a predetermined pose.
